(19) United States
(12) Plant Patent Application Publication
(10) Pub. No.: US 2013/0318663 P1
(43) Pub. Date: Nov. 28, 2013

(54) Plum tree named 'LRP40A'

(50) ;

(71) Applicant: Michael T. Malone, Havelock North (NZ)

(72) Inventor: Michael T. Malone, Havelock North (NZ)

(73) Assignee: The New Zealand Institute for Plant and Food Research Limited, Auckland (NZ)

(21) Appl. No.: 13/506,924

(22) Filed: May 24, 2012

(51) Int. Cl. A01H 5/00

(52) U.S. Cl. PLT/184

(57) ABSTRACT

A new and distinct plum variety is described. The variety results from selection among a population of seedlings derived from collecting seed from open pollinated fruit. Seedlings generated from the open pollination were planted out at Lawn Road, Havelock North, Hawke's Bay, New Zealand. In 2003, ‘LRP40A’ was identified as having potential as a new variety and was propagated for further trials. ‘LRP40A’ was selected for its desirable tree and fruit characteristics, and the timing of the harvest.

### GENUS AND SPECIES OF PLANT CLAIMED

Prunus salicina

### VARIETY DENOMINATION

‘LRP40A’

### BACKGROUND TO THE INVENTION

Seedlings obtained from collecting seed from open pollinated fruit were planted out at Lawn Road, Havelock North, Hawke's Bay, New Zealand in 2000. In March 2003, ‘LRP40A’ was identified as having potential as a new variety. Later in 2003, ‘LRP40A’ was first asexually propagated by budding onto ‘Golden Queen’ (not patented) peach seedling rootstock. ‘Golden Queen’ seedlings are a standard plum rootstock in New Zealand. The trees were planted out in Hawke's Bay during the southern hemisphere winter of 2004. The resulting plants propagated true to type, demonstrating that the characteristics of the new variety are stable and transmitted without change through succeeding generations.

### SUMMARY OF THE INVENTION

Under the New Zealand growing conditions ‘LRP40A’ is distinguished from varieties of common knowledge such as ‘Royal Star’ (U.S. Plant Pat. No. 7,192), ‘Omega’ (not patented) and its sibling ‘LRP40/205’ by the following characteristics:

The fruit of ‘Royal Star’ are cordate in shape with a truncate base whereas the fruit of ‘LRP40A’ is oblate in shape with a slightly depressed base and typically ripens approximately two weeks later. The skin colour of ‘Royal Star’ fruit is darker than that of ‘LRP40A’ while the flesh colour is lighter.

The fruit of ‘Omega’ ripens significantly earlier than that of ‘LRP40A’, early February and late March to early April respectively.

The fruit of ‘LRP40A’ are larger, cordate in shape, moderately asymmetric with a lightly mottled skin colour while the fruit of ‘LRP40A’ is oblate in shape and slightly asymmetric with a darker more solid skin colour.

### BRIEF DESCRIPTION OF FIGURES

The accompanying photographs show typical specimens of the, foliage, flowers and fruit of the new variety as depicted in colours as nearly true as is reasonably possible to make the same in a colour illustration of this character.

FIG. 1 shows fruit of ‘LRP40A’ on a two year old tree in the field (A and B).

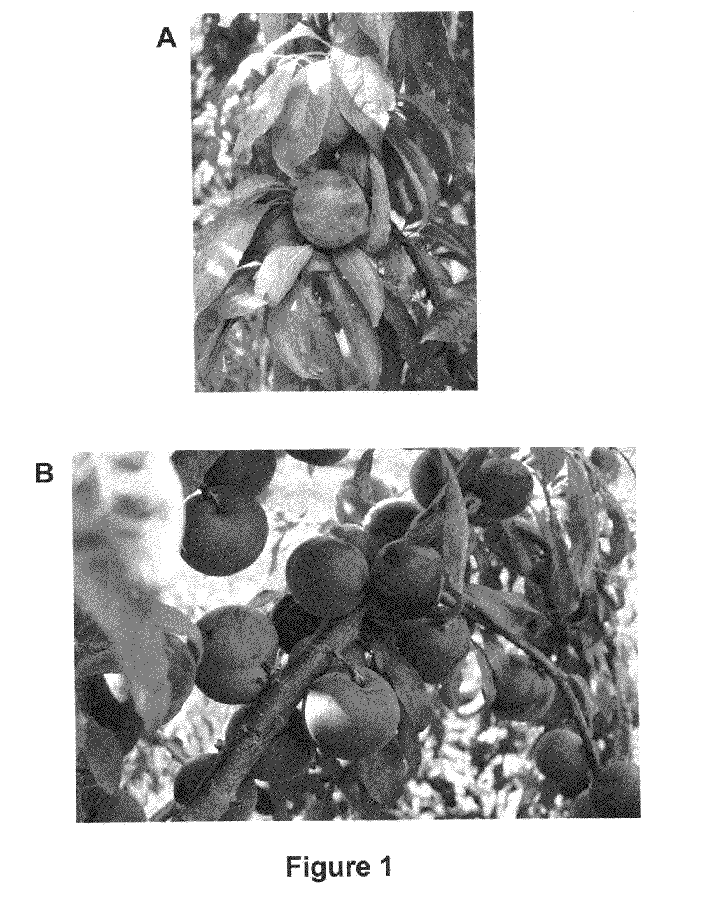

FIG. 2 shows fruit of ‘LRP40A’ (A) whole and (B) longitudinal section

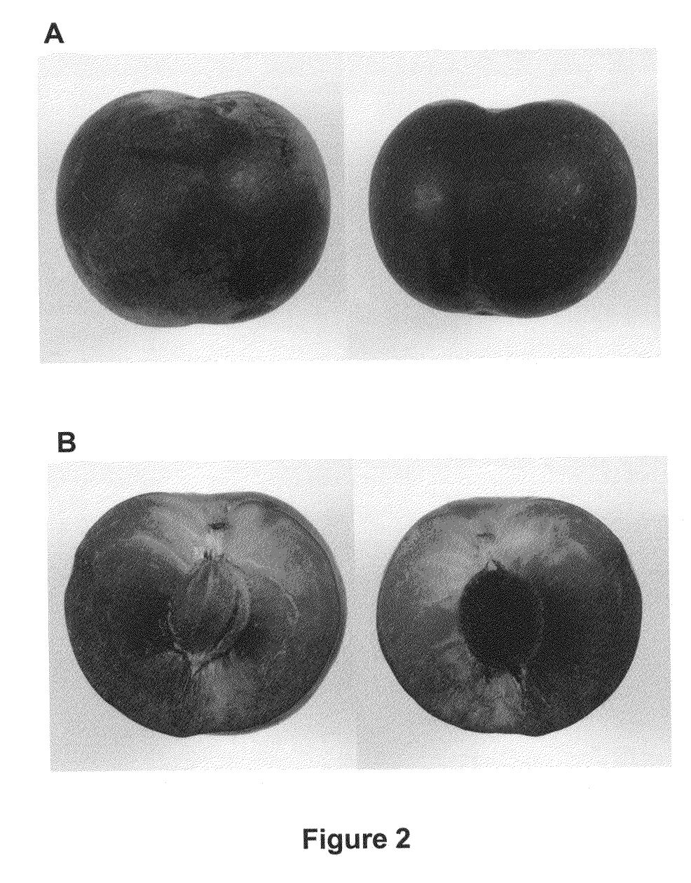

FIG. 3 shows ‘LRP40A’ (A) flowers at full bloom, (B) fully expanded leaf upper side, and (C) fully expanded leaf lower side.

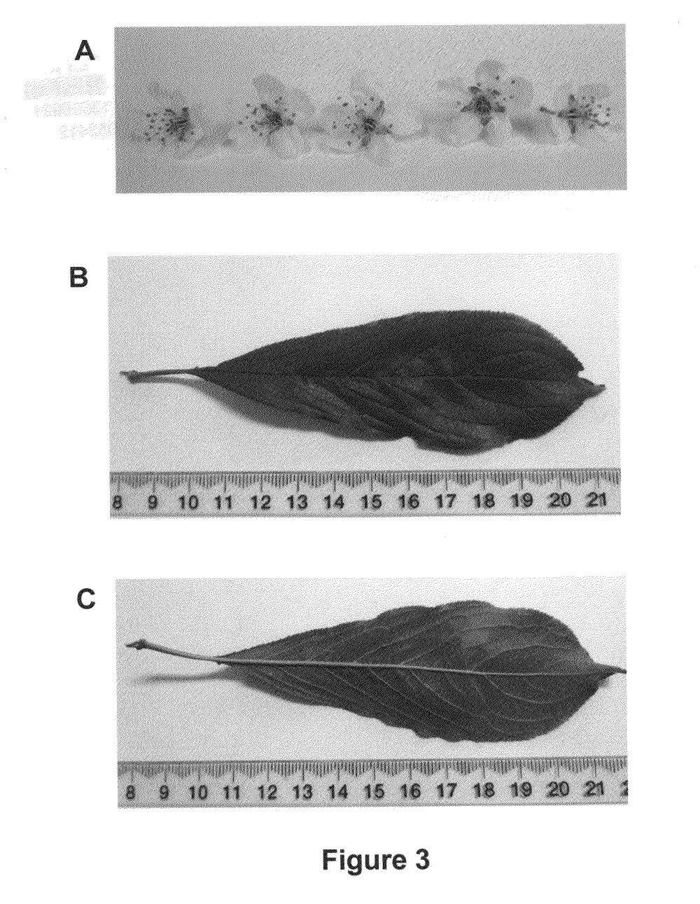

### DETAILED DESCRIPTION OF THE VARIETY

The following is a brief description of the new variety with colour terminology in accordance with The Royal Horticultural Society Colour Charts (R.H.S.C.C.) 2001 edition. The specimens described were grown in Hawke's Bay, New Zealand. The observations were made of the 2010-2012 seasons on mature trees which ranged in age between two and seven years old. Trees were grown under standard orchard practices.
Tree: considered to be a small-medium sized tree, ‘LRP40A’ is low vigour. ‘LRP40A’ scions are compatible with ‘Golden Queen’ peach seedlings which are the standard plum rootstock in New Zealand. Tree health is considered to be good under Hawke's Bay conditions in New Zealand with average yield of 50 kg per tree which is considered to be excellent under New Zealand conditions, with a similar precocity to commercially grown varieties in the area.
Trunk: average circumference was measured to be approximately 215 mm, measured approximately 200 mm above the graft union. The trunk was coloured near grey-brown N199B and near brown 200B, branches were coloured near brown N200A with near greyed-orange 165A lenticles on both the trunk and branches.
Leaves: the leaves examined were fully expanded and harvested from physiologically mature trees. The overall shape of the leaves was oblanceolate with a cuspidate tip, an acuminate base and serrulate margins. Leaves are clustered on spurs and singular on young branches. The colour of the upper side of the leaf was near green 137A and moderately glossy, while the lower side was near green 137C-D. The reticulate venation was coloured near yellow-green 145C extending to near green 145B on the underside at the end of the petiole. The average length of the blade was approximately 109.7 mm with an average width of approximately 38.6 mm.
Petiole: the average petiole length of fully expanded leaves was 20.0 mm with an average width of 2.0 mm. The colour of the upper side was near yellow-green 144A. The colour of the underside is described above.
Flowers: flower observations were made at full bloom which occurred mid to late September in Hawke's Bay, New Zealand. Flowers had a subtle fragrance. Flowers were clustered in groups of between one and three flowers.
Corolla: five petals per flower, with an average length of 10.0 mm and average width of 8.0 mm. Petals are predominantly free with some touching or slightly overlapping. The petal shape was observed to be broadly obovate with slightly undulate margins. The petals were near white N155A-B.
Stamens: an average of 30 per flower. Filaments were an average length of 6.8 mm and translucent white in colour. The dorsifixed anthers were near yellow-orange 14A in colour.
Carpel: pistil length was on average 8.5 mm in length.
Pedicel: average length was 5.2 mm and width 1.1 mm. Coloured near yellow-green 144C.
Calyx: five sepals per flower, average length of 3.0 mm and width 2.6 mm with an obtuse apex and smooth margin. Coloured near yellow-green 144B.
Fruit: harvested late March early April in Hawke's Bay, New Zealand depending on the season. Fruit are borne on both spurs and long shoots.
Size: fruit at harvest maturity were on average 118.54 g. Average height was 53.3 mm, width (lateral) 58.4 mm, and width (ventral) 63.1 mm.
Shape: fruit were slightly asymmetric, and oblate in shape, with a shallow suture, and a slightly depressed base.
Skin: fruit surface is smooth with a medium bloom. Background colour near red 53A and 45D.
Over colour: covers 95-99% of the fruit surface in a solid flush with a colour near greyed purple N187B-C with bloom, and near greyed purple 187A, near greyed purple N186C, and near purple N77A in the darkest areas with near red-purple 59A in lighter areas. Lenticels were coloured near yellow-orange 20B.
Flesh: fine grained and consistently dense, with a medium firmness. Colour is near greyed purple 185B and red-purple 60A in darkest areas and near red 45C in the lightest areas. The stone cavity colour is near greyed purple 187B and N77A.
Stone: elliptic in shape with an obtuse apex. Average dry weight was 1.6 g, with an average height of 20.6 mm, width (lateral) 16.2 mm, and width (ventral) 11.3, with a narrow stalk end average width 2.5 mm. Stone is clingstone and has a colour near greyed orange 165 D.
Peduncle: average length was 17.0 mm and width 3.0 mm. Coloured near greyed orange 166C.
Flavour: Flavour is sweet with low acidity.
Use: fresh eating, potential for processing
Keeping quality: Fruit stores for over 8 weeks at 0° C.

1. A new and distinct plum plant substantially as illustrated and described.
